(19) United States
(12) Patent Application Publication
(10) Pub. No.: US 2006/0155368 A1
(43) Pub. Date: Jul. 13, 2006

(54) Self-expandable stent

(76) Inventors: Kyoung-Min Shin, Seoul (KR); Jin-Hong Kim, Kyunggi-do (KR)

Correspondence Address: JORDAN AND HAMBURG LLP, 122 EAST 42ND STREET, SUITE 4000, NEW YORK, NY, 10168, US

(21) Appl. No.: 10/542,182

(22) PCT Filed: Jul. 29, 2003
(86) PCT No.: PCT/KR03/01513

§ 371 Date: Jul. 13, 2005

(51) Int. Cl. A61F 2/06

(52) U.S. Cl. 623/1.31

(57) ABSTRACT

The object of this invention is to provide a self-expandable stent, made of a shape-memory alloy and used for insertion in a narrow or blocked target portion of the contracted gullet having a lesion so as to open the target portion. The stent includes a coated primary unit having a hollow cylindrical body fabricated by knitting a shape-memory alloy wire to make a net structure, with both an enlarged diameter part and a coat layer. An uncoated secondary unit surrounds the hollow cylindrical body of the coated primary unit. The secondary unit has a hollow cylindrical body fabricated by knitting a superelastic shape-memory alloy wire to make a net structure having diamond-shaped meshes. The stent is prevented from being undesirably removed from a narrow or blocked target portion of the gullet having a lesion.

### TECHNICAL FIELD

The present invention relates to self-expandable stents made of a shape-memory alloy and used for insertion in a narrow or blocked target portion of a contracted gullet having a lesion so as to open the target portion of the gullet and, more particularly, to a self-expandable stent, designed to reliably maintain its desired position within a narrow or blocked target portion of the contracted gullet having a lesion for a desired lengthy period of time regardless of outside pressure caused by, for example, coughing or ingestion.

### BACKGROUND ART

As well known to those skilled in the medical field, it is very difficult or almost impossible for patients to ingest food when their gullets are blocked or contracted due to esophagostenosis caused by, for example, cancer.

In such a case, the ingestion may be accomplished through a tube, which passes through the abdomen to reach the stomach of a patient. Otherwise, it is necessary to open the narrow or blocked portion of the gullet through a surgical operation.

However, the installation of a tube passing through the abdomen causes severe pain to the patient, in addition to inconveniences to the family members of the patient. During a surgical operation for opening the narrow or blocked portion of the gullet, the skin of a patient is cut over a large area prior to removal of the lesion from the contracted gullet, and so the operation undesirably leaves a large and ugly scar on the skin and mars the appearance of the patient. The surgical operation also undesirably forces a patient to spend a lengthy period of time for recovery; nevertheless, it sometimes fails to accomplish its desired operational effect.

In an effort to overcome such problems experienced in conventional surgical operations or installation of ingestion tubes, an insertion of a balloon in a narrow or blocked portion of the contracted gullet to open the portion has been proposed and used. In order to install such a balloon in a desired portion of the contracted gullet, a balloon catheter tube is inserted into the target portion having the lesion prior to expanding the balloon set in the target portion to open the portion.

However, such an insertion of the balloon in the gullet is problematic in that the balloon only maintains its function for three or four months after the insertion, then allows the gullet to be contracted again after the lapse of such a period. Such an insertion of a balloon thus undesirably forces a patient to undergo repeated insertion of new balloons, in addition to paying additional money.

In order to solve the problems experienced by the use of such a balloon, the inventor of this invention proposed a stent, made of a shape-memory alloy and used for insertion in a target portion of the contracted gullet having a lesion. This stent was designed to almost permanently maintain its function within the target portion of the gullet, and was applied to KIPO under the Korean Patent Application No. 98-13572.

As shown in FIGS. 1 and 2, the conventional stent proposed by the inventor of this invention is fabricated by knitting a superelastic shape-memory alloy wire having a diameter of 0.1˜0.5 mm to make a hollow cylindrical stent body 2 having a net structure with a plurality of diamond-shaped meshes 3.

In order to allow the stent to effectively maintain its position within a target portion of the contracted gullet, an enlarged diameter part 5 is formed at each end of the hollow cylindrical stent body 2. Due to the enlarged diameter parts 5, the stent is reliably held in the target portion of the contracted gullet for a desired lengthy period of time without being undesirably removed from the target portion regardless of outside pressure.

When it is desired to insert the stent in a target portion of the contracted gullet having a lesion, the stent in a contracted configuration is primarily inserted into the target portion. Once the stent is inserted into a narrow or blocked target portion of the contracted gullet as shown in FIG. 3, the stent elastically expands, due to superelasticity of the shape-memory alloy, to open the target portion of the gullet.

However, the above-mentioned stent is fabricated by knitting a superelastic shape-memory alloy wire to make a hollow cylindrical stent body 2 having a net structure with the diamond-shaped meshes 3, and so the tumor cells of the gullet may infiltrate into the interior of the hollow body 2 through the meshes 3 to undesirably make the interior become narrow or blocked. In addition, when a patient ingests food through the mouth, food may come into frictional contact with the injured tissue of the lesion, thus causing severe pain to the patient during ingestion.

In an effort to overcome such problems, the stent is provided with a coat layer 7 on the external surface of the body 2 for externally covering the sidewall of the body 2 and preventing both an infiltration of the tumor cells into the interior of the body 2 and an undesired contact of food with the injured tissue of the lesion during ingestion.

However, the stent with such a coat layer 7 is problematic in that the stent may be movable within the slippery gullet due to smoothness of the coat layer 7, and so the stent may be removed from the target portion of the gullet when it is influenced by outside pressure caused by, for example, coughing or ingestion. When the stent is removed from a target portion of the contracted gullet, it is necessary to precisely adjust the position of the stent within the gullet or completely remove the existing stent prior to reinserting a new stent in the target portion of the gullet. This undesirably causes pain to a patient, in addition to forcing the patient to pay additional money.

### DISCLOSURE OF THE INVENTION

Accordingly, the present invention has been made keeping in mind the above problems occurring in the prior art, and an object of the present invention is to provide a self-expandable stent, which is designed to prevent both an infiltration of the tumor cells into the interior of the hollow stent set within a narrow or blocked target portion of the contracted gullet having a lesion and an undesired contact of food with the injured tissue of the lesion during ingestion, and which reliably maintains its desired position within a target portion of the gullet for a desired lengthy period of time regardless of outside pressure caused by, for example, coughing or ingestion, thus maintaining its function almost permanently.

In order to accomplish the above object, the present invention provides a self-expandable stent, which includes a coated primary unit having a hollow cylindrical body fabricated by knitting a shape-memory alloy wire to make a net structure having a plurality of diamond-shaped meshes, with both an enlarged diameter part formed at each end of the body and a coat layer formed on the external surface of the primary unit to cover the sidewall of the primary unit. The stent also includes an uncoated secondary unit surrounding the hollow cylindrical body of the coated primary unit. This secondary unit has a hollow cylindrical body fabricated by knitting a superelastic shape-memory alloy wire to make a net structure having a plurality of diamond-shaped meshes. The stent is thus prevented from being undesirably removed from a narrow or blocked target portion of the gullet having a lesion.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a side view of a self-expandable stent proposed by the inventor of this invention in the prior art;

FIG. 2 is a front view of the stent of FIG. 1;

FIG. 3 is a view, showing the stent of FIG. 1 set within the gullet of the body;

FIG. 4 is a side view of a self-expandable stent in accordance with the preferred embodiment of the present invention;

FIG. 5 is a front view of the stent of FIG. 4; and

FIG. 6 is a view, showing the stent of FIG. 4 set within the gullet of the body.

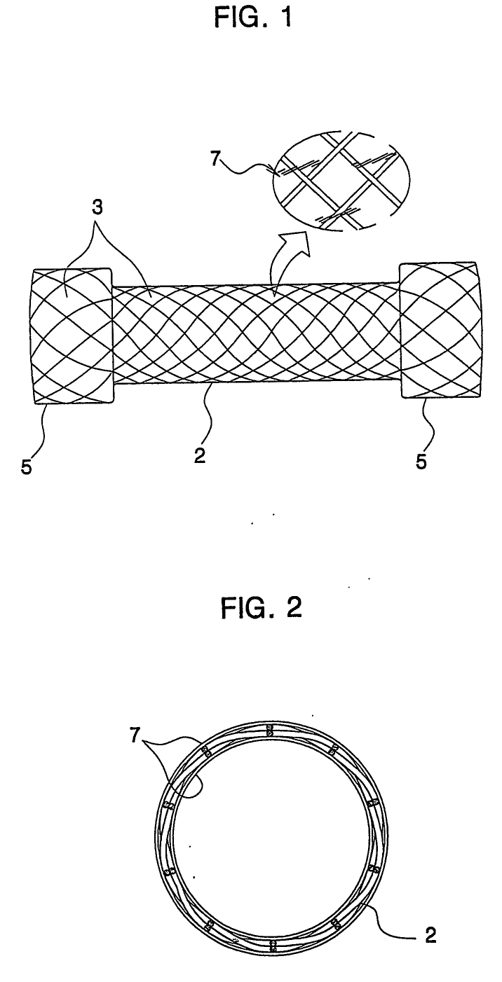

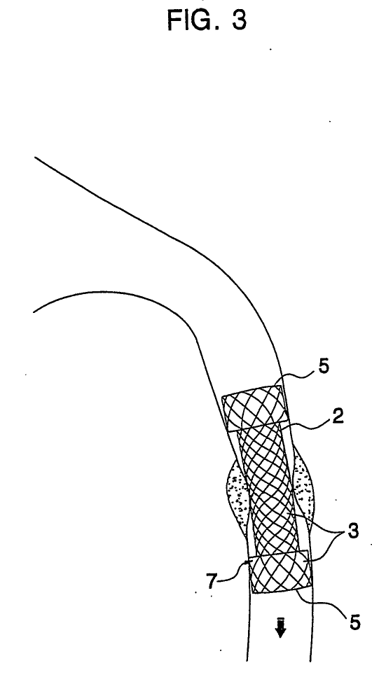

### BEST MODE FOR CARRYING OUT THE INVENTION

Reference now should be made to the drawings, in which the same reference numerals are used throughout the different drawings to designate the same or similar components.

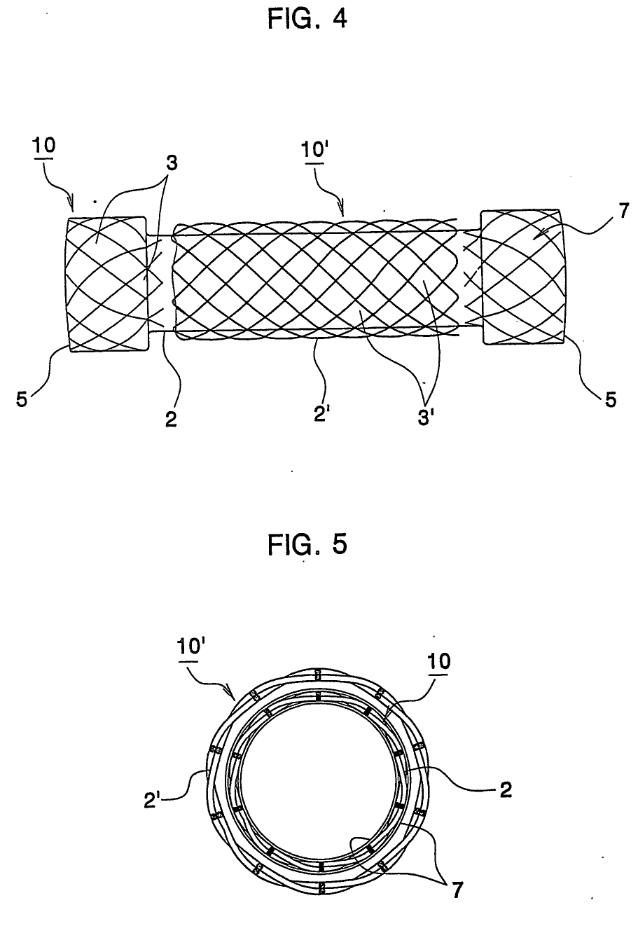

FIG. 4 is a side view of a self-expandable stent in accordance with the preferred embodiment of the present invention. FIG. 5 is a front view of the stent of FIG. 4. As shown in the drawings, the self-expandable stent of this invention comprises two units: a coated primary unit 10 and an uncoated secondary unit 10′. The coated primary unit 10 comprises a hollow cylindrical body 2 fabricated by knitting a shape-memory alloy wire to make a net structure having a plurality of diamond-shaped meshes 3. An enlarged diameter part 5 is formed at each end of the body 2, while a coat layer 7 is formed on the external surface of the primary unit 10 to externally cover the sidewall of the unit 10. The construction of the primary unit 10 remains the same as that the conventional stent proposed by the inventor of this invention.

The uncoated secondary unit 10′ surrounds the hollow cylindrical body 2 of the coated primary unit 10. This secondary unit 10′ comprises a hollow cylindrical body 2′ fabricated by knitting a superelastic shape-memory alloy wire to make a net structure having a plurality of diamond-shaped meshes 3′.

In the present invention, the two units 10 and 10′ are preferably integrated into a single body through, for example, a sewing process in order to prevent an undesired separation of them from each other.

In the drawings, the reference numeral 20 denotes the lesion of the contracted gullet, in which the stent of this invention is inserted.

The operational effect of the stent of this invention will be described herein below.

Prior to insertion of the stent in a narrow or blocked target portion of the contracted gullet having the lesion 20, it is necessary to measure the position, length and size of the lesion 20 and the inner diameter of the target portion using a separate fluoroscopic instrument.

After the measurement of the lesion 20, a coated primary unit 10 is prepared. In such a case, it is necessary to make the coated primary unit 10 longer than the length of the lesion 20. Particularly, the length between the inside edges of the two enlarged diameter parts 5 of the unit 10 must be longer than the length of the lesion 20 so as to allow the two enlarged diameter parts 5 to be positioned outside the opposite ends of the lesion 20.

The coated primary unit 10 is designed to have a diameter larger than the normal diameter of the gullet by about 10% ˜30%, thus securing a sufficient passage in the narrow or blocked target portion of the gullet having the lesion 20.

After the preparation of the coated primary unit 10, an uncoated secondary unit 10′ is fitted over the middle portion of the primary unit 10 between the two enlarged diameter parts 5 prior to being integrated with the primary unit 10 into a single structure through, for example, a sewing process.

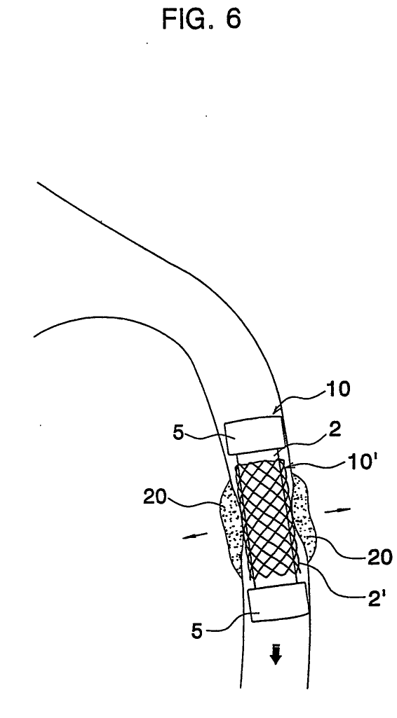

The stent, having the primary and secondary units 10 and 10′, is inserted in the target portion having the lesion 20. Such an insertion of the stent in the target portion of the contracted gullet is shown in FIG. 6.

In order to insert the stent in the target portion, the stent is primarily set in a separate stent inserting device (not shown) while being contracted in its radial direction to reduce its diameter.

When the stent is contracted as described above, the size of the diamond-shaped meshes 3 and 3′ of the two units 10 and 10′ is reduced, thus remarkably reducing the volume of the stent. It is thus possible to set the stent in the stent inserting device.

Thereafter, the stent insertion device is inserted into the gullet to reach the target portion having the lesion 20 prior to pushing the contracted stent to remove the stent from the inserting device. Once the stent is set in the narrow or blocked target portion having the lesion 20, the stent elastically expands to bias the lesion 20 outward in a radial direction due to superelasticity of the two units 10 and 10′ made of the shape-memory alloy, and desirably opens the target portion of the gullet.

In the stent set within the target portion having the lesion 20, the coated primary unit 10 prevents both an infiltration of the tumor cells into the interior of the stent and an undesired contact of food with the injured tissue of the lesion 20 during ingestion.

On the other hand, the uncoated secondary unit 10′ comes into close contact with the tissue of the lesion 20 at a position outside the coated primary unit 10, thus almost completely preventing an undesired removal of the stent from the target portion.

Due to the uncoated secondary unit 10′ fitted over the coated primary unit 10, the stent of this invention reliably maintains the passage of the gullet having the lesion 20 for a desired lengthy period of time without being undesirably removed from the target portion regardless of outside pressure caused by, for example, coughing or ingestion.

### INDUSTRIAL APPLICABILITY

As described above, the present invention provides a self-expandable stent, designed to reliably maintain its desired position within a narrow or blocked target portion of the contracted gullet having a lesion for a desired lengthy period of time regardless of outside pressure caused by, for example, coughing or ingestion. The stent of this invention comprises a coated primary unit and an uncoated secondary unit fitted over the primary unit. Both units are made of a shape-memory alloy, and are integrated into a single structure through, for example, a sewing process. When the stent is inserted in a narrow or blocked target portion of the contracted gullet having a lesion, the coated primary unit prevents both an infiltration of the tumor cells into the interior of the stent and an undesired contact of food with the injured tissue of the lesion during ingestion. In addition, the uncoated secondary unit comes into close contact with the tissue of the lesion at a position outside the coated primary unit, thus almost completely preventing an undesired removal of the stent from the target portion.

Although a preferred embodiment of the present invention has been described for illustrative purposes, those skilled in the art will appreciate that various modifications, additions and substitutions are possible, without departing from the scope and spirit of the invention as disclosed in the accompanying claims.

1. A self-expandable stent, comprising:
a coated primary unit having a hollow cylindrical body fabricated by knitting a shape-memory alloy wire to make a net structure having a plurality of diamond-shaped meshes, with both an enlarged diameter part formed at each end of the body and a coat layer formed on an external surface of the primary unit to externally cover a sidewall of the unit; and
an uncoated secondary unit surrounding the hollow cylindrical body of the coated primary unit, the secondary unit having a hollow cylindrical body fabricated by knitting a superelastic shape-memory alloy wire to make a net structure having a plurality of diamond-shaped meshes,
wherein the stent is prevented from being undesirably removed from a narrow or blocked target portion of the gullet having a lesion.
